(19) United States
(12) Patent Application Publication
(10) Pub. No.: US 2022/0270753 A1
(43) Pub. Date: Aug. 25, 2022

(54) APPLICATION FOR SELF-GOVERNED CLINICAL VALIDATION, VERIFICATION, AND REGISTRATION

(71) Applicant: Heroic Faith Medical Science Co., Ltd., New Taipei City (TW)

(72) Inventors: Fushun HSU, New Taipei City (TW); Fu Ji TSAI, New Taipei City (TW); Yuan Ren CHENG, New Taipei City (TW)

(21) Appl. No.: 17/735,662

(22) Filed: May 3, 2022

Related U.S. Application Data
(63) Continuation of application No. PCT/US2020/058928, filed on Nov. 4, 2020.
(60) Provisional application No. 62/929,962, filed on Nov. 4, 2019.

(51) Int. Cl. G16H 40/67; A61B 5/00; A61B 7/00; A61B 7/04; G16H 50/20

(52) U.S. Cl. CPC G16H 40/67; A61B 5/7221; G16H 50/20; A61B 7/04; A61B 7/003

(57) ABSTRACT

Introduced here is a decentralized computing system that is designed to efficiently connect various stakeholders involved in the healthcare system. An application that runs on the decentralized computing system may be responsible for performing activities in a secure manner to accelerate the accumulation and sharing of information among the various stakeholders. These activities may be governed by iterative protocols (e.g., blockchain protocols) that are implemented by the application using the decentralized system.

### CROSS-REFERENCE TO RELATED APPLICATIONS

This application is a continuation of International Application No. PCT/US2020/58928, filed on Nov. 4, 2020, which claims priority to U.S. Provisional Application No. 62/929,962, filed on Nov. 5, 2019, each of which is incorporated by reference herein in its entirety.

### TECHNICAL FIELD

Various embodiments pertain to an application that is designed to derive shareable healthcare-related insights.

### BACKGROUND

Validation, verification, and registration are important steps in the development and then deployment of medical devices and software to be used as a medical device. The terms “Software as a Medical Device” and “SaMD” refer to software that is intended to be used for medical purposes and that perform these purposes without being part of a medical device. For convenience, such software may simply be referred to as “medical software.”

There are difficulties in gathering the necessary information for validation, verification, and registration, however. These difficulties include issues with obtaining the clinical data needed to establish (or ensure compliance with) the Good Clinical Practice (GCP) standard and the informed consent of keyholders, as well as storing patient health information (also referred to as “protected health information” or “personal health information”) in an accessible-yet-secure manner. These are significant obstacles in the development and deployment of medical devices and medical software.

### BRIEF DESCRIPTION OF THE DRAWINGS

FIG. 1A includes a top perspective view of an input unit for an electronic stethoscope system.

FIGS. 1B-C include bottom perspective views of the input unit, which includes a structural body having a distal portion and a proximal portion.

FIG. 2 includes a cross-sectional side view of an input unit for an electronic stethoscope system.

FIG. 3 illustrates how one or more input units can be connected to a hub unit to form an electronic stethoscope system.

FIG. 4 is a high-level block diagram illustrating exemplary components of an input unit and a hub unit of an electronic stethoscope system.

FIG. 5 illustrates how a decentralized system differs from a centralized system.

FIG. 6 illustrates various stakeholders in the healthcare system.

FIG. 7 includes an example of an application designed to facilitate sharing of information amongst these stakeholders that may run on a decentralized system.

FIG. 8 illustrates several exemplary features of an application able to facilitate communication with various stakeholders in the healthcare system.

FIGS. 9A-D include several examples of smart contracts.

FIG. 10 includes a high-level illustration of a process in which information is shared amongst multiple stakeholders by an application.

FIG. 11 includes a high-level illustration of a process by which data generated by an electronic stethoscope system is shared with stakeholders having an interest in a patient.

FIG. 12 depicts an example workflow illustrating how data may be shared amongst various members of the healthcare system.

FIG. 13 depicts a flow diagram of a process for sharing data generated by a medical device with an entity that has an interest in the health of a patient.

FIG. 14 depicts a flow diagram of a process for distributing information regarding breathing abnormalities of a patient to entities that have an interest in the health of the patient.

FIG. 15 depicts a flow diagram of a process for sharing diagnostically relevant insights with stakeholders involved in triaging, diagnosing, or treating a patient.

FIG. 16 depicts an example of a workflow implemented by an application that runs on a decentralized system.

FIG. 17 is a block diagram illustrating an example of a processing system in which at least some operations described herein can be implemented.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications

### DETAILED DESCRIPTION

Introduced here is a decentralized computing system (or simply “decentralized system”) that is designed to efficiently connect various stakeholders involved in the healthcare system. As further discussed below, an application (also referred to as a “platform”) that runs on the decentralized system may be responsible for performing activities in a secure manner to accelerate the accumulation and sharing of information among the various stakeholders.

These activities may be governed by iterative protocols (e.g., blockchain protocols) that are implemented by the application using the decentralized system. As an example, assume that the application obtains an audio file that includes the respiratory sounds of an individual (also referred to as a “patient”). In such a scenario, the application may derive a healthcare-related insight by examining the audio file. This healthcare-related insight may be shared by the application with entities such as medical facilities (e.g., hospitals and clinics) and insurance providers to facilitate the diagnostic process.

For the purpose of illustration, the application may be described in the context of facilitating development and/or deployment of an electronic stethoscope system able to generate audio files that include the respiratory sounds of patients. However, those skilled in the art will recognize that the technology described herein could be used to facilitate development and/or deployment of other medical devices and medical software.

Embodiments may be described with reference to particular medical devices, computing devices, and networks. However, those skilled in the art will recognize that the features are similarly applicable to other medical devices, computing devices, and networks. For example, while the application may be described as configured to obtain and then examine audio files that contain respiratory sounds, the application could be configured to obtain then examine digital images (e.g., of the eye, lungs, etc.). Thus, the high-level approach to improving communication between various stakeholders in the healthcare system may be implemented regardless of the type of analysis performed by the application.

Moreover, while embodiments may be described in the context of computer-executable instructions, aspects of the technology could be implemented via software, firmware, or hardware. As an example, aspects of the application may be implemented on an inference server to simplify deployment of computer-implemented models (or simply “models”) at scale. Such an approach may allow models (e.g., for discovering breathing abnormalities in respiratory sounds) to be deployed from various frameworks and storage mediums (e.g., local memory or cloud-based memory).

### Terminology

Brief definitions of terms, abbreviations, and phrases used throughout the application are given below.

The terms “connected,” “coupled,” or any variant thereof is intended to include any connection or coupling between two or more elements, either direct or indirect. The connection or coupling can be physical, logical, or a combination thereof. For example, objects may be electrically or communicatively connected to one another despite not sharing a physical connection.

The term “module” may be used to refer broadly to components implemented via software, firmware, or hardware. Generally, modules are functional components that generate output(s) based on input(s). A computer program may include one or more modules. Thus, a computer program may include multiple modules responsible for completing different tasks or a single module responsible for completing all tasks.

Overview of Electronic Stethoscope System

Electronic stethoscope systems can be designed to simultaneously monitor sounds originating from within a living body (or simply “body”) under examination and the ambient environment. An electronic stethoscope system can include one or more input units that are connected to a hub unit. Each input unit may have a resonator and/or a diaphragm designed to direct acoustic sound waves toward at least one microphone configured to produce audio data indicative of internal sounds. These microphone(s) may be referred to as “auscultation microphones.” Moreover, each input unit may include at least one microphone configured to produce audio data indicative of sounds external to the body under examination. These microphone(s) may be referred to as “ambient microphones” or “environmental microphones.”

For the purpose of illustration, an “ambient microphone” may be described as capable of producing audio data indicative of “ambient sounds.” However, these “ambient sounds” generally include a combination of sounds produced by three different sources: (1) sounds originating from within the ambient environment (e.g., environmental noises); (2) sounds leaked through the resonator; and (3) sounds the penetrate the body under examination. Examples of ambient sounds include sounds originating directly from the structural body of the input unit (e.g., scratching by the finger or chest) and low-frequency environmental noises that penetrate the structural body of the input unit.

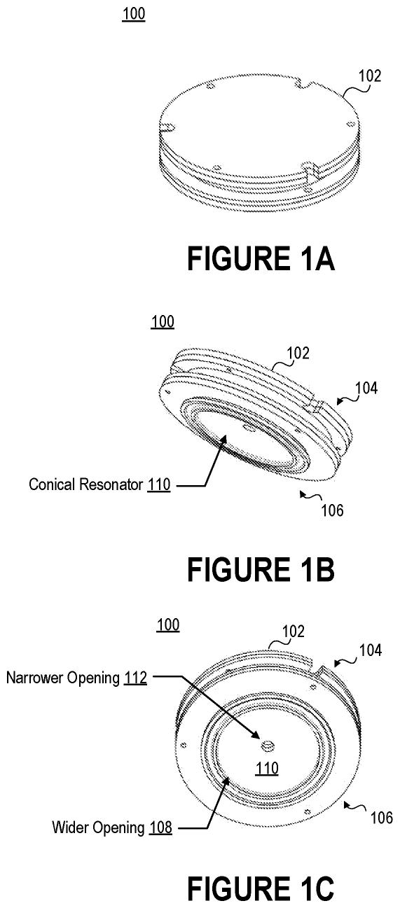

FIG. 1A includes a top perspective view of an input unit 100 for an electronic stethoscope system. For convenience, the input unit 100 may be referred to as a “stethoscope patch,” though, as further described below, the input unit may only include a subset of the components necessary for auscultation. The input unit 100 may also be referred to as a “chestpiece” since it will often be affixed to the chest of a body. However, those skilled in the art will recognize that the input unit 100 may be affixed to other parts of the body as well (e.g., the abdomen or the back).

As further described below, the input unit 100 can collect acoustic sound waves representative of biological activities within a body under examination, convert the acoustic sound waves into an electrical signal, and then digitize the electrical signal (e.g., for easier transmission, to ensure higher fidelity, etc.). The input unit 100 can include a structural body 102 comprised of metal, such as stainless steel, aluminum, titanium, or another suitable metal alloy. To make the structural body 102, molten metal will typically be die casted and then either machined or extruded into the appropriate form.

In some embodiments, the input unit 100 includes a casing that inhibits exposure of the structural body 102 to the ambient environment. For example, the casing may prevent contamination, improve cleanability, etc. Generally, the casing encapsulates substantially all of the structural body 102 except for the conical resonator disposed along its bottom side. The conical resonator is described in greater depth below with respect to FIGS. 1B-C. The casing may be comprised of silicon rubber, polypropylene, polyethylene, or any other suitable material. Moreover, in some embodiments, the casing includes an additive whose presence limits microbial growth, ultraviolet (UV) degradation, etc.

FIGS. 1B-C include bottom perspective views of the input unit 100, which includes a structural body 102 having a distal portion 104 and a proximal portion 106. To initiate an auscultation procedure, an individual (e.g., a medical professional, such as a physician or a nurse) can secure the proximal portion 106 of the input unit 100 against the surface of a body under examination. The proximal portion 106 of the input unit 100 can include the wider opening 108 of a conical resonator 110. The conical resonator 110 may be designed to direct acoustic sound waves collected through the wider opening 108 toward a narrower opening 112, which may lead to an auscultation microphone. Conventionally, the wider opening 108 is approximately 30-50 millimeters (mm), 35-45 mm, or 38-40 mm. However, because the input unit 100 described here may have automatic gain control functionality, smaller conical resonators may be used. For example, in some embodiments, the wider opening 108 is less than 30 mm, 20 mm, or 10 mm. Thus, the input units described herein may be able to support a wide variety of conical resonators having different sizes, designed for different applications, etc.

With regard to the terms “distal” and “proximal,” unless otherwise specified, the terms refer to the relative positions of the input unit 100 with reference to the body. For example, in referring to an input unit 100 suitable for fixation to the body, “distal” can refer to a first position close to where a cable suitable for conveying digital signals is connected to the input unit 100 and “proximal” can refer to a second position close to where the input unit 100 contacts the body.

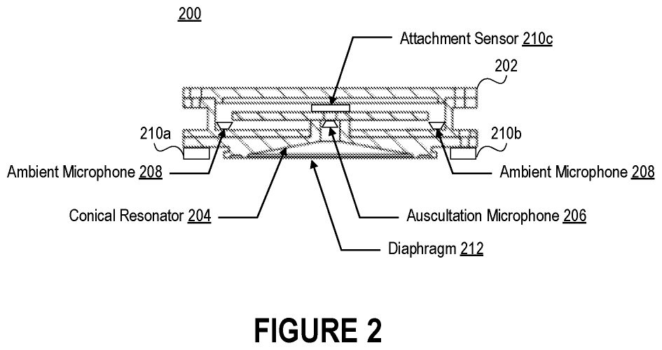

FIG. 2 includes a cross-sectional side view of an input unit 200 for an electronic stethoscope system. Often, the input unit 200 includes a structural body 202 having an interior cavity defined therein. The structural body 202 of the input unit 200 may have a conical resonator 204 designed to direct acoustic sound waves toward a microphone residing within the interior cavity. In some embodiments, a diaphragm 212 (also referred to as a “vibration film”) extends across the wider opening (also referred to as the “outer opening”) of the conical resonator 204. The diaphragm 212 can be used to listen to high-pitch sounds, such as those often produced by the lungs. The diaphragm 212 can be formed from a thin plastic disk comprised of an epoxy-fiberglass compound or glass fibers.

To improve the clarity of acoustic sound waves collected by the conical resonator 204, the input unit 200 may be designed to simultaneously monitor sounds originating from different locations. For example, the input unit 200 may be designed to simultaneously monitor sounds originating from within a body under examination and sounds originating from the ambient environment. Thus, the input unit 200 may include at least one microphone 206 (referred to as an “auscultation microphone”) configured to produce audio data indicative of internal sounds and at least one microphone 208 (referred to as an “ambient microphone”) configured to produce audio data indicative of ambient sounds. Each of the auscultation and ambient microphones includes a transducer able to convert acoustic sound waves into an electrical signal. Thereafter, the electrical signal may be digitized prior to transmission to a hub unit. Digitization enables the hub unit to readily clock or synchronize the signals received from multiple input units. Digitization may also ensure that the signal received by the hub unit from an input unit has a higher fidelity than would otherwise be possible.

These microphones may be omnidirectional microphones designed to pick up sound from all directions or directional microphones designed to pick up sounds coming from a specific direction. For example, the input unit 200 may include auscultation microphone(s) 206 oriented to pick up sounds originating from a space adjacent to the outer opening of the conical resonator 204. In such embodiments, the ambient microphone(s) 208 may be omnidirectional or directional microphones. As another example, a set of ambient microphones 208 could be equally spaced within the structural body 202 of the input unit 200 to form a phased array able to capture highly-directional ambient sounds to reduce noise and interference. Accordingly, the auscultation microphone(s) 206 may be arranged to focus on the path of incoming internal sounds (also referred to as the “auscultation path”), while the ambient microphone(s) 208 may be arranged to focus on the paths of incoming ambient sounds (also referred to as the “ambient paths”).

Conventionally, electronic stethoscopes subjected electrical signals indicative of acoustic sound waves to digital signal processing (DSP) algorithms responsible for filtering undesirable artifacts. However, such action may suppress nearly all of the sound within certain frequency ranges (e.g., 100-800 Hz), thereby greatly distorting internal sounds of interest (e.g., those corresponding to heartbeats, inhalations, or exhalations). Here, however, a processor can employ a noise cancellation algorithm that separately examines the audio data generated by the auscultation microphone(s) 206 and the audio data generated by the ambient microphone(s) 208. More specifically, the processor may parse the audio data generated by the ambient microphone(s) 208 to determine how, if at all, the audio data generated by the auscultation microphone(s) 206 should be modified. For example, the processor may discover that certain digital features should be amplified (e.g., because they correspond to internal sounds), diminished (e.g., because they correspond to ambient sounds), or removed entirely (e.g., because they represent noise). Such a technique can be used to improve the clarity, detail, and quality of sound recorded by the input unit 200. For example, application of the noise cancellation algorithm may be an integral part of the denoising process employed by an electronic stethoscope system that includes at least one input unit 200.

For privacy purposes, neither the auscultation microphone(s) 206 nor the ambient microphone(s) 208 may be permitted to record while the conical resonator 204 is directed away from the body. Thus, in some embodiments, the auscultation microphone(s) 206 and/or the ambient microphone(s) 208 do not begin recording until the input unit 200 is attached to body. In such embodiments, the input unit 200 may include one or more attachment sensors 210a-c that are responsible for determining whether the structural body 202 has been properly secured to the surface of the body.

The input unit 200 could include any subset of the attachment sensors shown here. For example, in some embodiments, the input unit 200 only includes attachment sensors 210a-b, which are positioned near the wider opening of the conical resonator 204. As another example, in some embodiments, the input unit 200 only includes attachment sensor 210c, which is positioned near the narrower opening (also referred to as the “inner opening”) of the conical resonator 204. Moreover, the input unit 200 may include different types of attachment sensors. For example, attachment sensor 210c may be an optical proximity sensor designed to emit light (e.g., infrared light) through the conical resonator 204 and then determine, based on the light reflected back into the conical resonator 204, the distance between the input unit 200 and the surface of the body. As another example, attachment sensors 210a-c may be audio sensors designed to determine, with the assistance of an algorithm programmed to determine the drop-off of a high-frequency signal, whether the structural body 202 is securely sealed against the surface of the body based on the presence of ambient noise (also referred to as “environmental noise”). As another example, attachment sensors 210a-b may be pressure sensors designed to determine whether the structural body 202 is securely sealed against the surface of the body based on the amount of applied pressure. Some embodiments of the input unit 200 include each of these different types of attachment sensors. By considering the output of these attachment sensor(s) 210a-c in combination with the aforementioned active noise cancellation algorithm, a processor may be able to dynamically determine the adhesion state. That is, the processor may be able to determine whether the input unit 200 has formed a seal against the body based on the output of these attachment sensor(s) 210a-c.

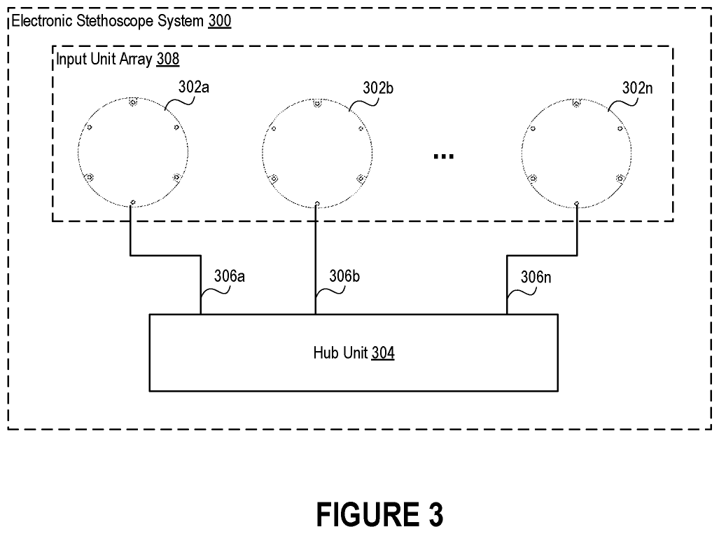

FIG. 3 illustrates how one or more input units 302a-n can be connected to a hub unit 304 to form an electronic stethoscope system 300. In some embodiments, multiple input units are connected to the hub unit 304. For example, the electronic stethoscope system 300 may include four input units, six input units, or eight input units. Generally, the electronic stethoscope system 300 will include at least six input units. Electronic stethoscope systems having multiple input units may be referred to as “multi-channel stethoscopes.” In other embodiments, only one input unit is connected to the hub unit 304. For example, a single input unit may be moved across the body in such a manner to simulate an array of multiple input units. Electronic stethoscope systems having one input unit may be referred to as “single-channel stethoscopes.”

As shown in FIG. 3, each input unit 302a-n can be connected to the hub unit 304 via a corresponding cable 306a-n. Generally, the transmission path formed between each input unit 302a-n and the hub unit 304 via the corresponding cable 306a-n is designed to be substantially free of interference. For example, electronic signals may be digitized by the input units 302a-n prior to transmission to the hub unit 304, and signal fidelity may be ensured by prohibiting the generation/contamination of electromagnetic noise. Examples of cables include ribbon cables, coaxial cables, Universal Serial Bus (USB) cables, High-Definition Multimedia Interface (HDMI) cables, RJ45 ethernet cables, and any other cable suitable for conveying a digital signal. Each cable includes a first end connected to the hub unit 304 (e.g., via a physical port) and a second end connected to the corresponding input unit (e.g., via a physical port). Accordingly, each input unit 302a-n may include a single physical port, and the hub unit 304 may include multiple physical ports. Alternatively, a single cable may be used to connect all of the input units 302a-n to the hub unit 304. In such embodiments, the cable may include a first end capable of interfacing with the hub unit 304 and a series of second ends, each of which is capable of interfacing with a single input unit. Such a cable may be referred to, for example, as a “one-to-two cable,” “one-to-four cable,” or “one-to-six cable” based on the number of second ends.

When all of the input units 302a-n connected to the hub unit 304 are in an auscultation mode, the electronic stethoscope system 300 can employ an adaptive gain control algorithm programmed to compare internal sounds to ambient sounds. The adaptive gain control algorithm may analyze a target auscultation sound (e.g., normal breathing, wheezing, crackling, etc.) to judge whether an adequate sound level has been achieved. For example, the adaptive gain control algorithm may determine whether the sound level exceeds a predetermined threshold. The adaptive gain control algorithm may be designed to achieve gain control of up to 100 times (e.g., in two different stages). The gain level may be adaptively adjusted based on the number of input units in the input unit array 308, as well as the level of sound recorded by the auscultation microphone(s) in each input unit. In some embodiments, the adaptive gain control algorithm is programmed for deployment as part of a feedback loop. Thus, the adaptive gain control algorithm may apply gain to audio recorded by an input unit, determine whether the audio exceeds a preprogrammed intensity threshold, and dynamically determine whether additional gain is necessary based on the determination.

Because the electronic stethoscope system 300 can deploy the adaptive gain control algorithm during a post-processing procedure, the input unit array 308 may be permitted to collect information regarding a wide range of sounds caused by the heart, lungs, etc. Because the input units 302a-n in the input unit array 308 can be placed in different anatomical positions along the surface of the body (or on an entirely different body), different biometric characteristics (e.g., respiratory rate, heart rate, or degree of wheezing, crackling, etc.) can be simultaneously monitored by the electronic stethoscope system 300.

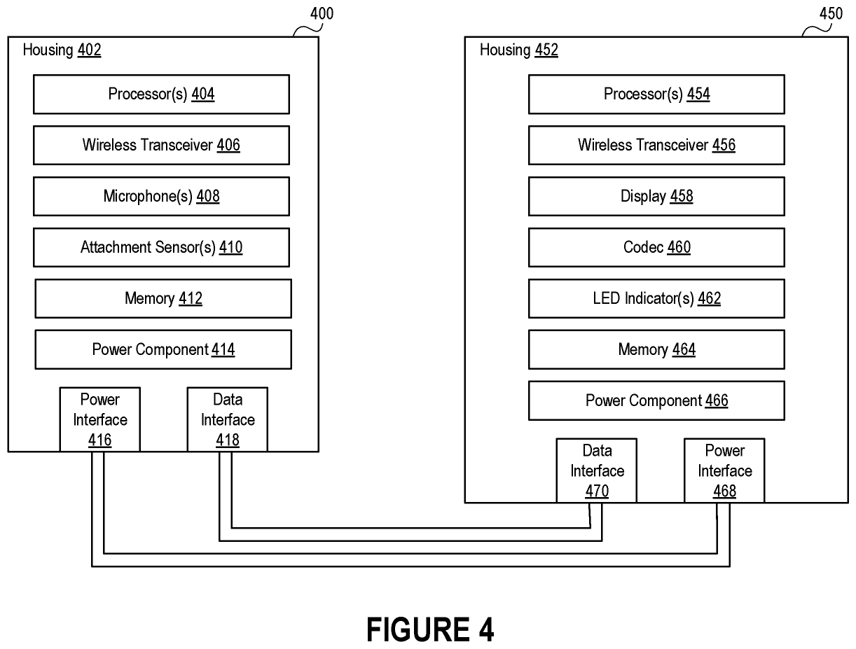

FIG. 4 is a high-level block diagram illustrating exemplary components of an input unit 400 and a hub unit 450 of an electronic stethoscope system. Embodiments of the input unit 400 and the hub unit 450 can include any subset of the components shown in FIG. 4, as well as additional components not illustrated here. For example, the input unit 400 may include a biometric sensor capable of monitoring a biometric characteristic of the body, such as perspiration (e.g., based on skin humidity), temperature, etc. Additionally or alternatively, the biometric sensor may be designed to monitor a breathing pattern (also referred to as a “respiratory pattern”), record electrical activity of the heart, etc. As another example, the input unit 400 may include an inertial measurement unit (IMU) capable of generating data from which gesture, orientation, or position can be derived. An IMU is an electronic component designed to measure the force, angular rate, inclination/tilt, and/or magnetic field of an object. Generally, an IMU includes accelerometer(s), gyroscope(s), magnetometer(s), or any combination thereof.

The input unit 400 can include one or more processors 404, a wireless transceiver 406, one or more microphones 408, one or more attachment sensors 410, a memory 412, and/or a power component 414 electrically coupled to a power interface 416. These components may reside within a housing 402 (also referred to as a “structural body”).

As noted above, the microphone(s) 408 can convert acoustic sound waves into an electrical signal. The microphone(s) 408 may include auscultation microphone(s) configured to produce audio data indicative of internal sounds, ambient microphone(s) configured to produce audio data indicative of ambient sounds, or any combination thereof. Audio data representative of values of the electrical signal can be stored, at least temporarily, in the memory 412. In some embodiments, the processor(s) 404 process the audio data prior to transmission downstream to the hub unit 450. For example, the processor(s) 404 may apply algorithms designed for digital signal processing, denoising, gain control, noise cancellation, artifact removal, feature identification, etc. In other embodiments, minimal processing is performed by the processor(s) 404 prior to transmission downstream to the hub unit 450. For example, the processor(s) 404 may simply append metadata to the audio data that specifies the identity of the input unit 400 or examine metadata already added to the audio data by the microphone(s) 408.

In some embodiments, the input unit 400 and the hub unit 450 transmit data between one another via a cable connected between corresponding data interfaces 418, 470. For example, audio data generated by the microphone(s) 408 may be forwarded to the data interface 418 of the input unit 400 for transmission to the data interface 470 of the hub unit 450. Alternatively, the data interface 470 may be part of the wireless transceiver 456. The wireless transceiver 406 could be configured to automatically establish a wireless connection with the wireless transceiver 456 of the hub unit 450. The wireless transceivers 406, 456 may communicate with one another via a bi-directional communication protocol, such as Near Field Communication (NFC), wireless Universal Serial Bus (USB), Bluetooth, Wi-Fi, a cellular data protocol (e.g., LTE, 3G, 4G, or 5G), or a proprietary point-to-point protocol.

The input unit 400 may include a power component 414 able to provide power to the other components residing within the housing 402, as necessary. Similarly, the hub unit 450 can include a power component 466 able to provide power to the other components residing within the housing 452. Examples of power components include rechargeable lithium-ion (Li-Ion) batteries, rechargeable nickel-metal hydride (NiMH) batteries, rechargeable nickel-cadmium (NiCad) batteries, etc. In some embodiments, the input unit 400 does not include a dedicated power component, and thus must receive power from the hub unit 450. A cable designed to facilitate the transmission of power (e.g., via a physical connection of electrical contacts) may be connected between a power interface 416 of the input unit 400 and a power interface 468 of the hub unit 450.

The power channel (i.e., the channel between power interface 416 and power interface 468) and the data channel (i.e., the channel between data interface 418 and data interface 470) have been shown as separate channels for the purpose of illustration only. Those skilled in the art will recognize that these channels could be included in the same cable. Thus, a single cable capable of carrying data and power may be coupled between the input unit 400 and the hub unit 450.

The hub unit 450 can include one or more processors 454, a wireless transceiver 456, a display 458, a codec 460, one or more light-emitting diode (LED) indicators 462, a memory 464, and a power component 466. These components may reside within a housing 452 (also referred to as a “structural body”). As noted above, embodiments of the hub unit 450 may include any subset of these components, as well as additional components not shown here. For example, some embodiments of the hub unit 450 include a display 458 for presenting information such as the respiratory status or heartrate of an individual under examination, a network connectivity status, a power connectivity status, a connectivity status for the input unit 400, etc. The display 458 may be controlled via tactile input mechanisms (e.g., buttons accessible along the surface of the housing 452), audio input mechanisms (e.g., voice commands), etc. As another example, some embodiments of the hub unit 450 include LED indicator(s) 462 for operation guidance rather than the display 458. In such embodiments, the LED indicator(s) 462 may convey similar information as the display 458. As another example, some embodiments of the hub unit 450 include a display 458 and LED indicator(s) 462.

Upon receiving audio data representative of the electrical signal generated by the microphone(s) 408 of the input unit 400, the hub unit 450 may provide the audio data to a codec 460 responsible for decoding the incoming data. The codec 460 may, for example, decode the audio data (e.g. by reversing encoding applied by the input unit 400) in preparation for editing, processing, etc. The codec 460 may be designed to sequentially or simultaneously process audio data generated by the auscultation microphone(s) in the input unit 400 and audio data generated by the ambient microphone(s) in the input unit 400.

Thereafter, the processor(s) 454 can process the audio data. Much like the processor(s) 404 of the input unit 400, the processor(s) 454 of the hub unit 450 may apply algorithms designed for digital signal processing, denoising, gain control, noise cancellation, artifact removal, feature identification, etc. Some of these algorithms may not be necessary if already applied by the processor(s) 404 of the input unit 400. For example, in some embodiments the processor(s) 454 of the hub unit 450 apply algorithm(s) to discover diagnostically relevant features in the audio data, while in other embodiments such action may not be necessary if the processor(s) 404 of the input unit 400 have already discovered the diagnostically relevant features. Alternatively, the hub unit 450 may forward the audio data to a destination (e.g., a diagnostic service running on a decentralized system) for analysis, as further discussed below. Generally, a diagnostically relevant feature will correspond to a pattern of values in the audio data matching a predetermined pattern-defining parameter. As another example, in some embodiments the processor(s) 454 of the hub unit 450 apply algorithm(s) to reduce noise in the audio data to improve the signal-to-noise (SNR) ratio, while in other embodiments these algorithm(s) are applied by the processor(s) 404 of the input unit 400.

In addition to the power interface 468, the hub unit 450 may include a power port. The power port (also referred to as a “power jack”) enables the hub unit 450 to be physically connected to a power source (e.g., an electrical outlet). The power port may be capable of interfacing with different connector types (e.g., C13, C15, C19). Additionally or alternatively, the hub unit may include a power receiver having an integrated circuit (“chip”) able to wirelessly receive power from an external source. The power receiver may be configured to receive power transmitted in accordance with the Qi standard developed by the Wireless Power Consortium or some other wireless power standard.

In some embodiments, the housing 452 of the hub unit 450 includes an audio port. The audio port (also referred to as an “audio jack”) is a receptacle that can be used to transmit signals, such as audio, to an appropriate plug of an attachment, such as headphones. An audio port typically includes, two, three, or four contacts that enable audio signals to be readily transmitted when an appropriate plug is inserted into the audio port. For example, most headphones include a plug designed for a 3.5-millimeter (mm) audio port. Additionally or alternatively, the wireless transceiver 456 of the hub unit 450 may be able to transmit audio signals directly to wireless headphones (e.g., via NFC, Bluetooth, etc.).

As noted above, the processor(s) 404 of the input unit 400 and/or the processor(s) 454 of the hub unit 450 can apply a variety of algorithms to support different functionalities. Examples of such functionalities include attenuation of lost data packets in the audio data, noise-dependent volume control, dynamic range compression, automatic gain control, equalization, noise suppression, and acoustic echo cancellation.

Each functionality may correspond to a separate module residing in a memory (e.g., memory 412 of the input unit 400 or memory 464 of the hub unit 450). Thus, the input unit 400 and/or the hub unit 450 may include an attenuation module, a volume control module, a compression module, a gain control module, an equalization module, a noise suppression module, an echo cancellation module, or any combination thereof.

Additional information on electronic stethoscope systems can be found in U.S. Pat. No. 10,555,717, which is incorporated by reference herein in its entirety.

### Overview of Application

Validation, verification, and registration are important steps in the development and then deployment of medical devices and medical software. There are difficulties in gathering the information needed for validation, verification, and registration, however. This is especially true now, as many entities have begun developing medical software that is designed to accompany a given medical device. As an example, the electronic stethoscope system discussed with reference to FIGS. 1A-4 may be supported by a diagnostic service able to provide analysis of audio data generated by the electronic stethoscope system.

Introduced here is a decentralized computing system (or simply “decentralized system”) that is designed to efficiently connect various stakeholders involved in the healthcare system. An application that runs on the decentralized system may be responsible for accelerating the accumulation and sharing among the various stakeholders so as to facilitate the rendering of services to individuals (also referred to as “patients”).

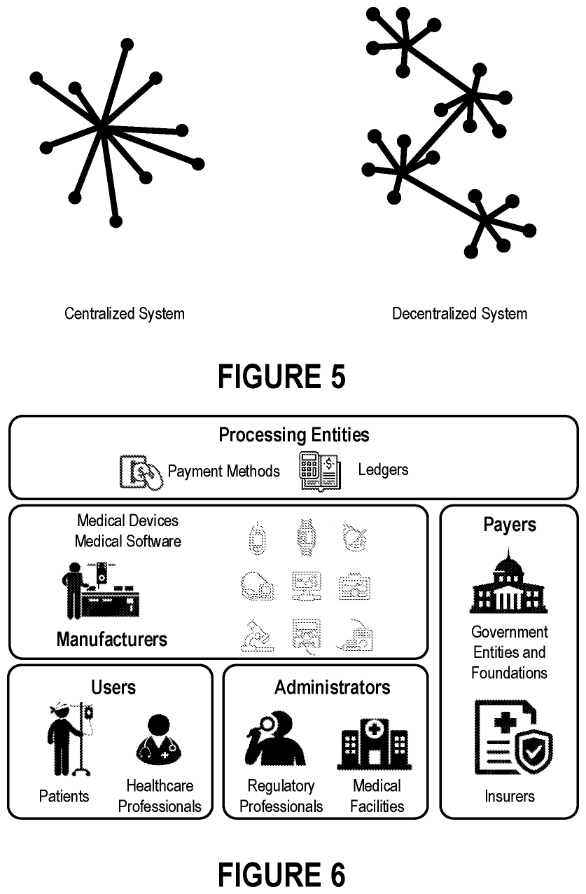

FIG. 5 illustrates how a decentralized system differs from a centralized system. The terms “centralized” and “decentralized” refer to the level of control over the system. In a centralized system, control is exerted by a single entity. In a decentralized system, there is no single controlling entity. Instead, control is shared among several entities. For example, the decentralized system described herein may be controlled by various stakeholders in the healthcare system. These stakeholders may include patients and healthcare professionals (collectively referred to as “users” of the application), manufacturers of medical devices and medical software, processing entities, regulatory professionals and medical facilities (collectively referred to as “administrators”), and payers such as insurers and government entities (e.g., the Centers for Medicare and Medicaid Services), as shown in FIG. 6.

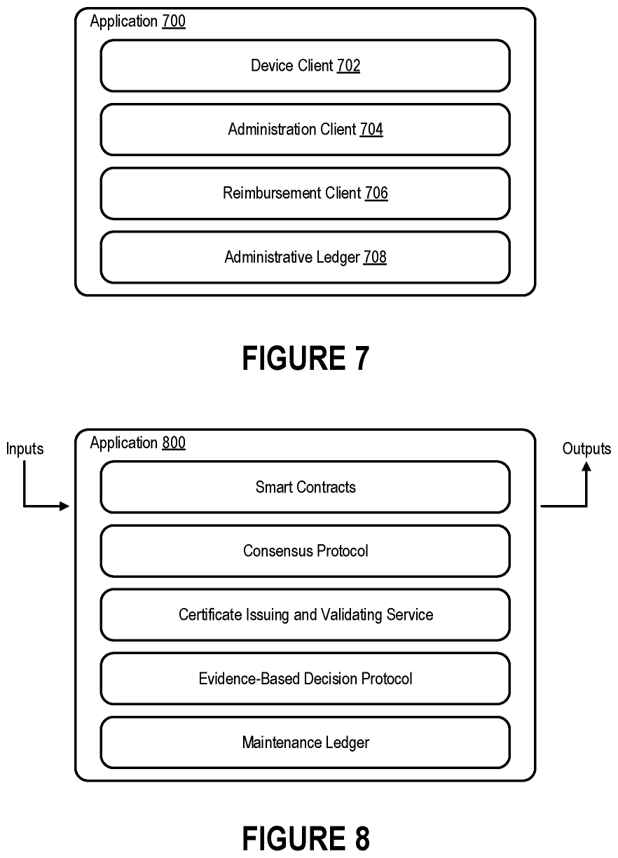

FIG. 7 includes an example of an application 700 designed to facilitate sharing of information amongst these stakeholders that may run on a decentralized system. The application 700 (also referred to as a “decentralized program” or “decentralized platform”) may comprise code that runs on a decentralized peer-to-peer network. In some embodiments, the application is incentivized through providing tokens to those stakeholders who validate the application 700.

As shown in FIG. 7, the application 700 may comprise a series of clients that may reside on different nodes in the decentralized system. For example, a device client 702 may reside on a medical device that is associated with a user (e.g., a patient or healthcare professional), while an administration client 704 may reside on a computing device that is managed by an administrator (e.g., a regulatory professional or medical facility, such as a hospital, clinic, or laboratory). A reimbursement client 706 may reside on a computing device that is managed by a payer (e.g., a government entity, foundation, or insurer). An administrative ledger 708, meanwhile, may reside on a computing device that is managed by a processing entity. Collectively, the device client 702, administration client 704, reimbursement client 706, and administrative ledger 708 support a diagnostic service offered by the application 700.

FIG. 8 illustrates several exemplary features of an application 800 able to facilitate communication with various stakeholders in the healthcare system. One key aspect of the application 800 is its ability to acquire input from stakeholders and then convey appropriate outputs to other stakeholders. To accomplish this, the application 800 may utilize smart contracts. The term “smart contract” refers to a computer program that is intended to automatically execute, control, or document relevant events according to the terms of an agreement. At a high level, smart contracts are automated (i.e., self-executing) contracts with instructions written in the underlying code that are executed only when certain conditions are met. Using smart contracts, the stakeholders mentioned above can exchange data in a trusted, conflict-free manner without relying on a third party. In some embodiments, smart contracts implemented or enforced by the application are stored on a blockchain. As such, those smart contracts may need to be validated much like a regular transaction, as further discussed below.

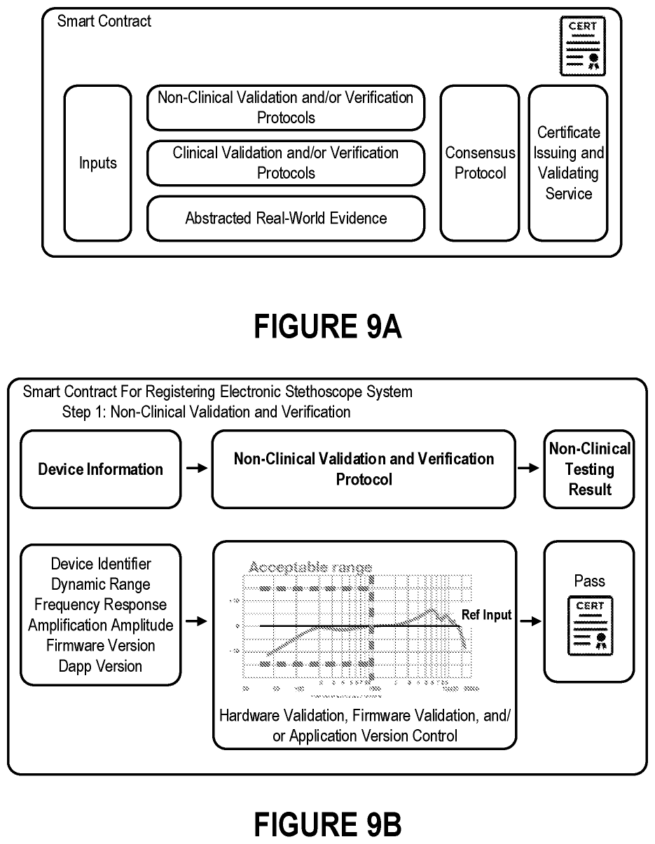

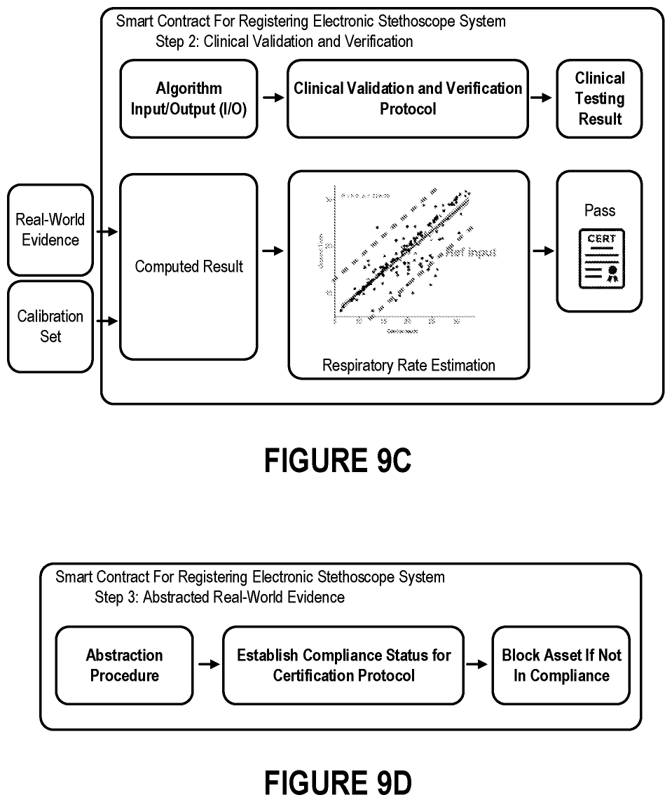

Note that smart contracts may also be used in the validation, verification, and registration of medical devices and medical software. Several examples of such smart contracts are shown in FIGS. 9A-D. As shown in FIG. 9A, based on the data provided as input, the smart contract may specify what steps, if any, should be performed in accordance with a non-clinical validation and verification protocol or a clinical validation and verification protocol. Alternatively, the smart contract may specify how to abstract, process, or convey the data in the form of real-world evidence. As further discussed below, performance of the terms of the smart contract may be governed by a consensus protocol and overseen by a certificate issuing and validating service.

FIG. 9B depicts an example of a smart contract programmed for registering a medical device (here, an electronic stethoscope system) in accordance with a non-clinical validation and verification protocol. As can be seen in FIG. 9B, when executed by an application, the smart contract may determine whether to permit registration based on information regarding the medical device. In the case of an electronic stethoscope system, this information may include dynamic range, frequency response, and amplification amplitude, as well as the firmware version and application (DApp) version. This information may be determined and/or derived through non-clinical testing of the medical device. The smart contract may require that this information conforms with predetermined threshold values or ranges. If the smart contract indicates that registration is appropriate, then the passage can be formalized with a digital certificate that is provisioned by the application. The digital certificate provisioned by the application may specify or indicate that non-clinical validation and verification was completed.

FIG. 9C depicts an example of a smart contract programmed for registering a medical device (here, an electronic stethoscope system) in accordance with a clinical validation and verification protocol. As can be seen in FIG. 9C, when executed by an application, the smart contract may determine whether to permit registration based on the accuracy of metrics that are either produced by the medical device or rely on data generated by the medical device. In the case of an electronic stethoscope system, the smart contract may require that respiratory rate be calculated with sufficient accuracy. Thus, the smart contract may require that an algorithm designed to produce an output indicative of the respiratory rate be applied to real-world evidence (e.g., data generated by the medical device) and a calibration set. This data may be provided via an application programming interface (API) or bulk data interface. The application may provision a digital certificate that specifies or indicates that clinical validation and verification was completed.

FIG. 9D depicts an example of a smart contract programmed for registering a medical device (here, an electronic stethoscope system) using abstracted real-world evidence. As can be seen in FIG. 9D, when executed by an application, the smart contract may determine whether to permit registration based on whether the medical device is in compliance with a certification protocol. Over time, data generated by the medical device may be abstracted and then examined in order to determine whether the data indicates compliance with the certification protocol. The medical device may be blocked so as to inhibit further use responsive to a determination that the medical device is not in compliance with the certification protocol. For example, the application may prevent further use of the medical device (e.g., by transmitting an instruction to cease operation to the device client), or the application may restrict communication by preventing further data to be uploaded and/or analyzed until the medical device is in compliance with the certification protocol.

Note that these smart contracts could be executed by the application in series. Thus, the smart contract in FIG. 9D may be executed following the successful completion of the smart contract in FIG. 9C, and the smart contract in FIG. 9C may be executed following the successful completion of the smart contract in FIG. 9B. Accordingly, the smart contracts in FIGS. 9B, 9C, and 9D may be implemented as sequential steps for registering a medical device such as an electronic stethoscope system. Each of these smart contracts may not be “enforced” until the preceding smart contract has been successfully completed. Thus, the smart contract shown in FIG. 9D may not be “enforced” until the smart contracts shown in FIGS. 9B-C have been successfully completed.

Another aspect of the application 800 is its ability to operate in compliance with a consensus protocol (or simply “protocol”) agreed upon by the various stakeholders. As discussed above, the application 800 may be spread across various nodes (e.g., computing devices) whose job is to verify and/or record transactions. Because any stakeholder could submit information to be recorded, it is important that there are rules in place that specify what information should be recorded and what information should be discarded. These rules are referred to as the “protocols” by which the application 800 operates. At a high level, the protocol defines the approach to verifying whether a transaction recorded by the decentralized platform 800 is true or not. The protocol may be fully defined before the diagnostic service is offered by the application 800, or the protocol may be altered while the diagnostic service is offered by the decentralized platform 800. Thus, the protocol could be adjusted while the decentralized platform 800 is “live.”

In some embodiments, the application 800 is configured to implement a certificate issuing and validating service in order to validate the various stakeholders. As an example, the application 800 may support and/or utilize a decentralized Public Key Infrastructure (PKI) in which a blockchain is used as a key-value storage. The term “blockchain,” as used herein, refers to a distributed digital ledger containing information that can be simultaneously used and shared within the decentralized system discussed above. Decentralized PKI eliminates dependence on certificate authorities for key management, relying instead on the decentralized system to maintain digital certificates (e.g., authorizing access to data to a given stakeholder).

Decisions made by the application 800 may be made in accordance with an evidence-based decision protocol. To make an evidence-based decision, the application 800 may consider various forms of evidence and then assess appropriateness of the evidence given the situation. Assume, for example, that the application 800 obtains audio data that includes the respiratory sounds of a patient for whom a diagnosis is to be recommended. In such a scenario, the application 800 may consider personal health information regarding the patient in addition to any insights derived from analyzing the audio data. The audio data and personal health information may be obtained from the same source (e.g., a device client) or different sources (e.g., a device client and an administration client).

As discussed above, transactions may be recorded by the application 800 on a distributed digital ledger. The distributed digital ledger may represent the backbone of the application 800, as it represents a consensus of information spread across multiple nodes (e.g., computing devices associated with different stakeholders).

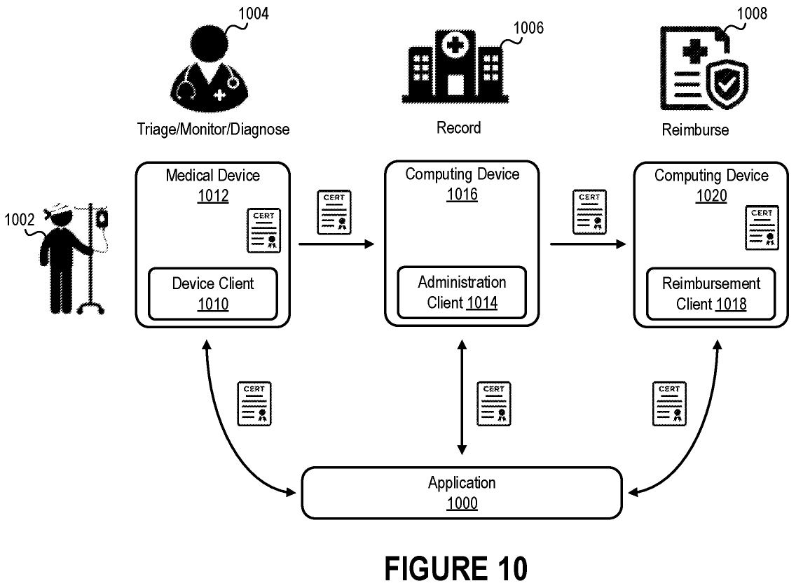

FIG. 10 includes a high-level illustration of a process in which information is shared amongst multiple stakeholders by an application 1000. Here, the multiple stakeholders include a patient 1002, medical professional 1004, medical facility 1006, and payer 1008. These stakeholders are involved in different aspects of the healthcare system. For example, the medical professional 1004 may be responsible for triaging, monitoring, or diagnosing the patient 1002, while the medical facility 1006 may be responsible for recording information regarding the patient 1002. Meanwhile, the payer 1008 may be responsible for reimbursing the medical professional 1004 and/or medical facility 1006 for services rendered.

As shown in FIG. 10, a device client 1010 may reside on a medical device 1012 that is employed by the medical professional 1004 to treat and/or diagnose the patient 1002. One example of a medical device is the electronic stethoscope system discussed above with reference to FIGS. 1A-4. The device client 1010 may provide data generated by the medical device 1012 to the application 1000. In some embodiments, the data is continually transferred to the application 1000 in real time (i.e., as the data is being generated). In other embodiments, the data is periodically transferred to the application 1000. For example, the device client 1010 may be configured to transmit the data in accordance with a predetermined schedule (e.g., every 15 minutes, 30 minutes, etc.), or the device client 1010 may be configured to transmit the data upon receiving an instruction to do so (e.g., following the conclusion of a diagnostic session conducted by the medical professional 1004).

Then, the application 1000 can determine, through the use of a smart contract, whether to permit access to the data to the medical facility 1006. If the application 1000 determines that the medical facility 1006 is authorized to access the data, then the data can be transferred to an administration client 1014 that resides on a computing device 1016 accessible to the medical facility 1006. The computing device 1016 may be, for example, a personal computer or computer server. Upon receiving the data through the administration client 1014, the medical facility 1006 may store the data in an electronic medical record that is associated with the patient 1002. The electronic medical record may also include data obtained from other sources (e.g., entered directly by the medical professional 1004).

Similarly, the application 1000 may determine, through the use of a smart contract, whether to permit access to the data to the payer 1008. If the application 1000 determines that the payer 1008 is authorized to access the data, then the data can be transferred to a reimbursement client 1018 that resides on a computing device 1020 accessible to the payer 1008. The computing device 1016 may be, for example, a personal computer or computer server. Upon receiving the data through the reimbursement client 1018, the payer 1008 may examine the data so as to determine whether reimbursement is appropriate.

While the embodiment shown in FIG. 10 is described in the context of sharing data generated by the medical device 1012, those skilled in the art will recognize that the process is similarly applicable if analysis of the data are shared in addition to, or instead of, the data. As an example, assume that the device client 1010 transfers data generated by the medical device 1012 to the application 1000 as discussed above. However, rather than transfer the data to the administration client 1014, the application 1000 may transfer analysis of the data to the administration client 1014. For instance, if the medical device 1012 is an electronic stethoscope system configured to generate audio data that includes respiratory sounds of the patient 1002, then the application 1000 (and, more specifically, a diagnostic service implemented by the application) may examine the audio data to identify breathing abnormalities and provide information regarding those breathing abnormalities to the administration client 1014. Similarly, the application 1000 may provide information regarding the data to the reimbursement client 1018. For instance, the application 1000 may provide, to the reimbursement client 1018, information regarding the activities performed by the medical professional 1004 rather than the data generated by the medical device 1012.

In some embodiments, the stakeholders involved in this process are verified by a certificate issuing and validating service implemented by the application 1000. As shown in FIG. 10, the certificate issuing and validating service may enable the stakeholders to exchange digital certificates (or simply “certificates”) with the application 1000 and one another for the purpose of establishing trust. The term “certificate,” as used herein, refers to an electronic document that is used to provide the ownership of a cryptographic key. At a high level, a certificate is representative of digital credentials that bind the identity of the owner (e.g., a stakeholder) to a pair of encryption keys (one public and one private) that can be used to encrypt data. A certificate includes information about the cryptographic key and information about its owner, and thus can be used in a decentralized system as a means of establishing authenticity.

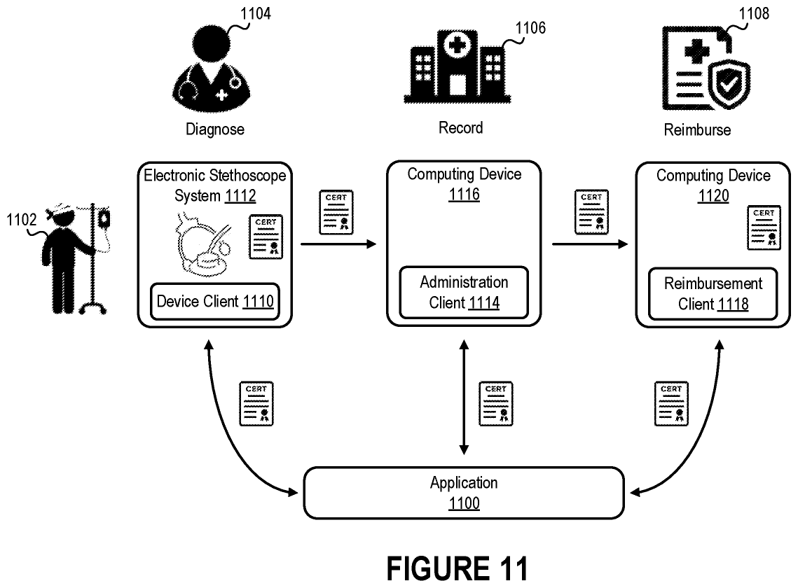

FIG. 11 includes a high-level illustration of a process by which data generated by an electronic stethoscope system 1112 is shared with stakeholders having an interest in a patient 1102. Initially, a medical professional 1104 may use the electronic stethoscope system 1112 in an effort to diagnose the patient 1102. Over the course of the diagnostic session, the electronic stethoscope system 1112 may generate audio data that includes the respiratory sounds produced by the patient 1102 over an interval of time. For example, the medical professional 1104 may use the electronic stethoscope system 1112 to record the respiratory sounds for 10-60 seconds, 10-30 seconds, or 10-15 seconds. Generally, the medical professional 1104 will seek to record at least three full breathing cycles (i.e., inhaling and then exhaling). A device client 1110 that resides on the electronic stethoscope system 1112 may be responsible for transmitting the audio data to an application 1100 for further analysis.

As mentioned above, the application 1100 may be configured to execute a diagnostic service upon receiving the audio data. For example, the application 1100 may apply, to the audio data, a model that is designed and trained to calculate the respiratory rate, identify breathing abnormalities, or recommend appropriate treatments. These outputs may be referred to as “analysis” of the audio data.

Thereafter, the application 1100 can determine whether to permit access to a stakeholder, such as a medical facility 1106 or payer 1108. Generally, this is accomplished through the use of smart contracts as discussed above. If the application 1100 determines that access is authorized for a given stakeholder, then the analysis and/or the audio data can be shared. In FIG. 11, for example, access is authorized for two stakeholders (i.e., the medical facility 1106 and payer 1108). The information that is appropriate for sharing may be specified in the smart contract corresponding to each stakeholder. For example, the smart contract that authorizes access to the medical facility 1106 may specify that the analysis of audio data should be provided to an administration client 1114 that resides on a computing device 1116 accessible to the medical facility 1106. As another example, the smart contract that authorizes access to the payer 1108 may specify that information regarding the activities performed by the medical professional 1104 should be provided to a reimbursement client 1118 that resides on a computing device 1120 accessible to the payer 1108. Such information could specify the type of medical device used for diagnosis, the name of the patient 1102, the name of the medical professional 1104, the name of the medical facility 1106 where diagnosing occurred, and the like.

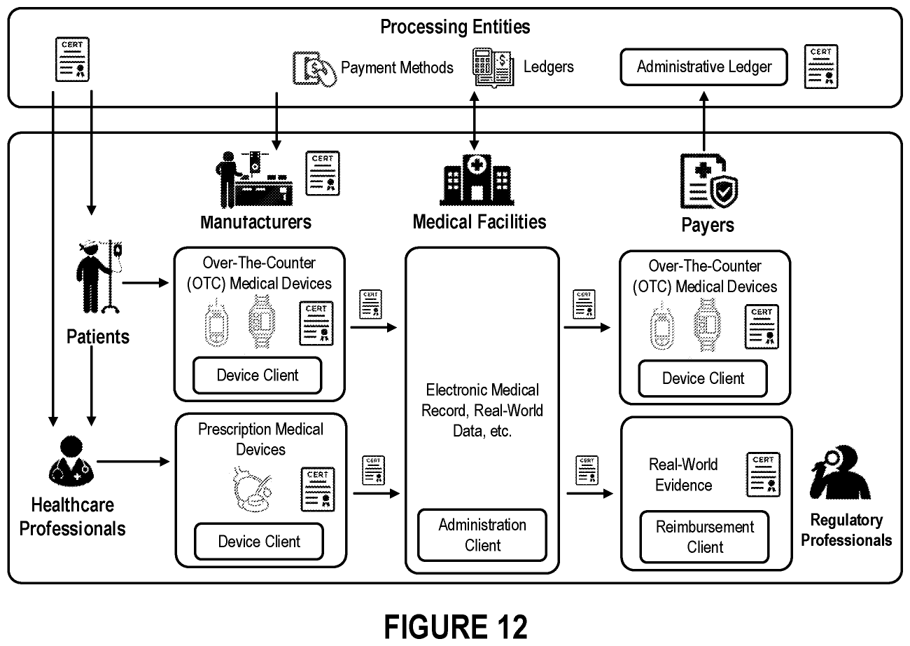

In FIGS. 10-11, the application is communicatively connected to a single device client, administration client, and reimbursement client. Those skilled in the art will recognize, however, that an application could include multiple device clients, administration clients, and reimbursement clients. FIG. 12 depicts an example workflow illustrating how data may be shared amongst various members of the healthcare system. Some members (e.g., medical professionals, medical facilities, and payers) may be intimately involved in providing care to patients, while other members (e.g., regulatory professionals) may be more focused on ensuring that care is provided in the appropriate manner (e.g., using properly validated medical devices).

Methodologies for Facilitating Sharing of Data Using an Application

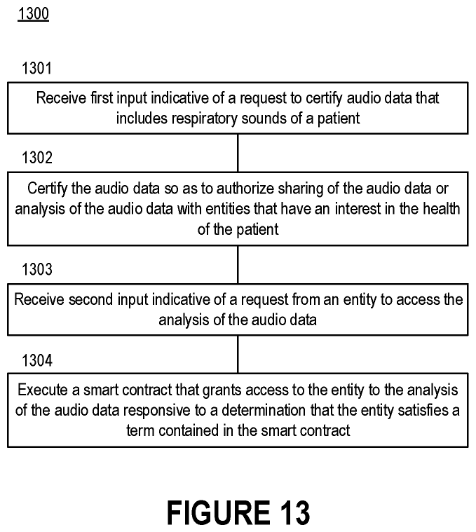

FIG. 13 depicts a flow diagram of a process 1300 for sharing data generated by a medical device with an entity that has an interest in the health of a patient. This process 1300 may be implemented by an application running on a decentralized system, as discussed above.

Initially, the application may receive first input indicative of a request to certify audio data that includes the respiratory sounds of a patient (step 1301). The first input may be received in various ways. For example, the first input may be obtained from a client (e.g., a device client) that resides on the medical device. As another example, the first input may be provided through an interface generated and/or supported by the application. Assume, for example, that the application is able to generate an interface accessible through a computer program, such as a mobile application, desktop application, or web browser. In such a scenario, an individual may seek certification by uploading the audio file to the application through the interface. The individual may be the patient or some other person (e.g., a medical professional or family member).

Then, the application can certify the audio data so as to authorize sharing of the audio data (or analysis of the audio data) with entities that have an interest in the health of the patient (step 1302). For example, the application may obtain information regarding the medical device responsible for generating the audio data and then examine the information to ensure that the medical device satisfies one or more criteria. Examples of such criteria include dynamic range, frequency response, amplification amplitude, firmware version, and application (DApp) version. Upon determining that the medical device satisfies the criteria, the application may produce the analysis of the audio file. As discussed above, the analysis may include information regarding respiratory rate, breathing abnormalities, and the like. As another example, the application may examine the audio data to ensure that the audio data meets one or more criteria. Examples of such criteria include a minimum duration, a minimum number of breathing cycles, a minimum signal-to-noise (SNR) ratio, and the like. Upon determining that the audio data satisfies the criteria, the application may produce the analysis of the audio file.

Thereafter, the application may receive second input indicative of a request from an entity to access the analysis of the audio data (step 1303). This second input may be received from a client that resides on a computing device separate from the medical device. The client could be an administration client associated with a medical facility or regulatory professional, or the client could be a reimbursement client associated with a government organization or insurer. The application can then execute a smart contract that grants access to the entity to the analysis of the audio data responsive to a determination that the entity satisfies a term in the smart contract. As an example, the smart contract may require that the entity have a valid digital certificate stored in, or accessible to, the decentralized system on which the application is running.

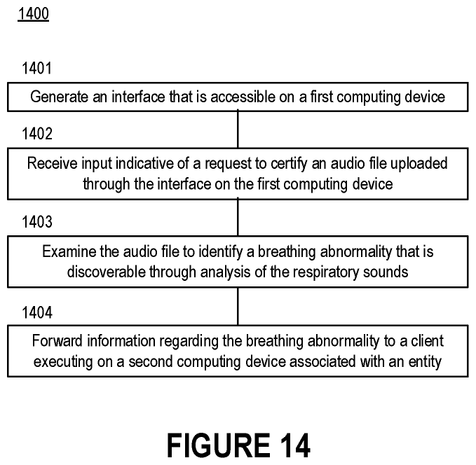

FIG. 14 depicts a flow diagram of a process 1400 for distributing information regarding breathing abnormalities of a patient to entities that have an interest in the health of the patient. Initially, an application can generate an interface that is accessible on a first computing device (step 1401). Then, the application can receive input indicative of a request to certify an audio file uploaded through the interface on the first computing device (step 1402). The audio file may include the respiratory sounds that are produced by a patient over an interval of time and recorded by an electronic stethoscope system. Meanwhile, the first computing device could be a mobile phone, tablet computer, personal computer, or mobile computer workstation (also referred to as a “mobile computer cart”). Generally, the type of computing device depends on the individual who accesses the interface. For example, if the patient herself uploads the audio file, then she may be more likely to do so through a mobile phone or tablet computer. However, if a medical professional uploads the audio file, then she may be more likely to do so through a personal computer or mobile computer workstation.

Thereafter, the application can examine the audio file to identify a breathing abnormality that is discoverable in the respiratory sounds (step 1403). For example, the application may apply an algorithm to the audio file that is trained to identify instances of wheezing, cracking, stridor, and rhonchi. The application can then forward information regarding the breathing abnormality to a client executing on a second computing device associated with an entity (step 1404). The entity may be a manufacturer of the medical device responsible for generating the audio file, a medical facility that is responsible for treating the patient, or an insurer involved in paying for treatment of the individual. While the first and second computing devices may both be representative of nodes in the decentralized system on which the application is running, the first and second computing devices need not be the same type of computing device. For example, the audio file may be uploaded through an interface accessible via a web browser or a mobile phone or personal computer, and the analysis may be forwarded to a client executing on a computer server.

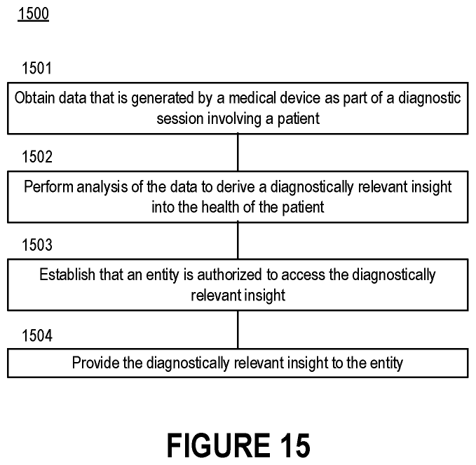

FIG. 15 depicts a flow diagram of a process 1500 for sharing diagnostically relevant insights with stakeholders involved in triaging, diagnosing, or treating a patient. Initially, an application can obtain data that is generated by a medical device as part of a diagnostic session involving a patient (step 1501). Generally, the diagnostic session is supervised by a medical professional, for example, within a medical facility such as a hospital or clinic. However, the diagnostic session could occur in another setting. For example, the patient may be able to complete the diagnostic session herself, for example, within her home. Meanwhile, the type of data will depend on the nature of the medical device. For example, if the medical device is an electronic stethoscope system, then the data may include audio content. As another example, if the medical device is an imaging instrument (e.g., a retinal camera or x-ray machine), then the data may include visual content in the form of digital images.

The application can then perform analysis of the data to derive a diagnostically relevant insight into the health of the patient (step 1502). As an example, the application may examine audio content to identify breathing abnormalities or cardiovascular abnormalities. As another example, the application may examine visual content to identify abnormal growths and lesions. In some embodiments, the application records evidence of the diagnostically relevant insight being derived as a transaction on a digital ledger maintained by the decentralized system on which the application runs.

Moreover, the application may establish that an entity is authorized to access the diagnostically relevant insight (step 1503). For example, the application may execute a smart contract that authorizes access to the entity responsive to a determination that the entity satisfies a term contained in the smart contract. Alternatively, the application may determine that the diagnostically relevant insight is to be shared with the entity based on information related to the patient (e.g., name or date of birth), diagnostic session (e.g., location, time, supervising medical professional), or medical device (e.g., manufacturer), and then confirm that a digital certificate establishing authenticity of the entity is valid. This digital certificate may be stored in the digital ledger maintained by the decentralized system.

Thereafter, the application can provide the diagnostically relevant insight to the entity (step 1504). For example, the application may forward the diagnostically relevant insight (or information regarding the diagnostically relevant insight) to a client that resides on a computing device accessible to the entity. In some embodiments, the application records evidence of the diagnostically relevant insight being provided to the entity as a transaction on the digital ledger maintained by the decentralized system.

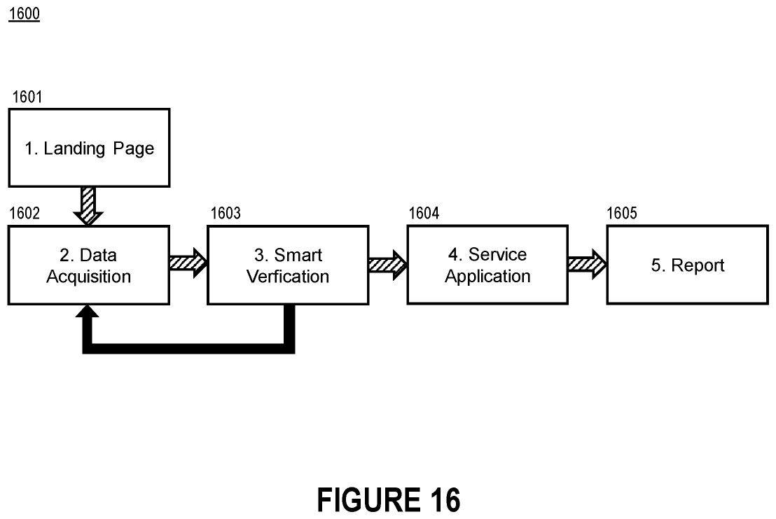

FIG. 16 depicts an example of a workflow 1600 implemented by an application that runs on a decentralized system. Initially, users may arrive at a landing page through which a diagnostic service (or simply “service”) offered by the application can be accessed (step 1601). The landing page may contain data entry prompts where the users can input medical data for the service to analyze. As an example, if the service is designed to analyze audio files (e.g., that include the respiratory sounds of patients), then the interface may include an upload graphical element (also referred to as an “upload button”) and progress bar indicating whether an audio file was successfully uploaded. In some embodiments, instructional materials (e.g., a video) are available through the landing page to assist users in recording and/or inputting medical data, or otherwise operating the service.

As discussed above, the medical data will be acquired by the service (step 1602) after a user has entered and/or uploaded the medical data in the requisite formats through the landing page. To ensure this is properly completed, the user may simply upload the medical data (e.g., by selecting an audio file), or the user may view instructional materials to ensure that the medical data is being properly uploaded.

Thereafter, the medical data input by the user can be verified (step 1603) via a decentralized process that takes place, partially or entirely, on the computing device used by the user to access the landing page. If verification fails, the user may be taken back to the previous step to make a second attempt to input the medical data. The approaches to decentralized verification may include, but are not limited to, log regression. Additionally or alternatively, the service may verify the quality of the medical data uploaded by the user against one or more evaluation parameters (e.g., defined by the manufacturer of the medical device used to generate the medical data).

If the medical data is determined to meet the evaluation parameter(s), then the medical data can be transmitted by the computing device to another computing device (e.g., a computer server) that is responsible for analyzing the medical data (step 1604) and generating appropriate reports (step 1605). As an example, the medical data may be transferred via the Internet to a computer server on which an algorithm driven through artificial intelligence (AI) or machine learning (ML) performs breathing sound identification and compiles reports. Reports can be communicated to the user, for example, via push notification, text message, email, and the like. Additionally or alternatively, reports may be available for download through a web browser or for online viewing only. Reports may include analysis or results as interpreted on behalf of the user, as well as recommended follow-up actions. For example, the report may recommend that, based on breathing abnormalities discovered in an audio file that contains the respiratory sounds of a patient, that the patient visit a healthcare provider.

Unless contrary to physical possibility, it is envisioned that the steps described above may be performed in various sequences and combinations. For example, multiple instances of the processes 1300, 1400, 1500 may be executed by the application as data pertaining to one patient is shared with a first set of entities while data pertaining to another patient is shared with a second set of entities. Other steps may also be included in some embodiments. For example, diagnostically relevant insights, such as the presence of breathing abnormalities, could be posted to an interface for review by a patient in addition to, or instead of, the medical professional responsible for diagnosing the patient.

Processing System

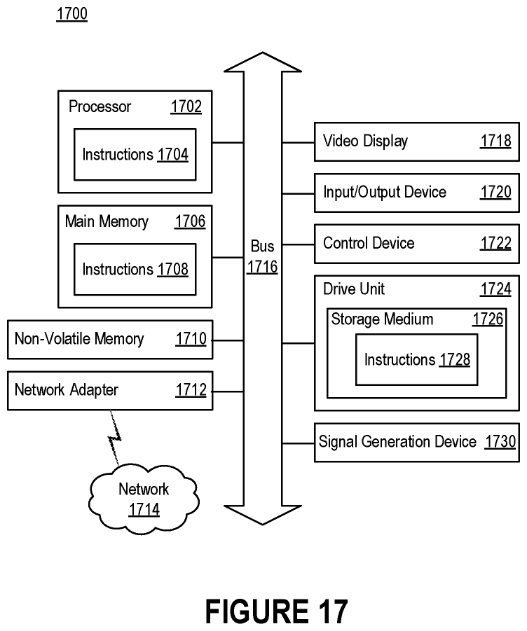

FIG. 17 is a block diagram illustrating an example of a processing system 1700 in which at least some operations described herein can be implemented. For example, components of the processing system 1700 may be hosted on a computing device that is representative of a node in a decentralized system on which an application runs. Examples of nodes include medical devices and other computing devices, such as mobile phones, tablet computers, personal computers, and computer servers.

The processing system 1700 may include a processor 1702, main memory 1706, non-volatile memory 1710, network adapter 1712 (e.g., a network interface), video display 1718, input/output device 1720, control device 1722 (e.g., a keyboard, pointing device, or mechanical input such as a button), drive unit 1724 that includes a storage medium 1726, or signal generation device 1730 that are communicatively connected to a bus 1716. The bus 1716 is illustrated as an abstraction that represents one or more physical buses and/or point-to-point connections that are connected by appropriate bridges, adapters, or controllers. The bus 1716, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or a bus compliant with Institute of Electrical and Electronics Engineers (IEEE) Standard 1394.

The processing system 1700 may share a similar computer processor architecture as that of a computer server, router, desktop computer, tablet computer, mobile phone, video game console, wearable electronic device (e.g., a watch or fitness tracker), network-connected (“smart”) device (e.g., a television or home assistant device), augmented or virtual reality system (e.g., a head-mounted display), or another electronic device capable of executing a set of instructions (sequential or otherwise) that specify action(s) to be taken by the processing system 1700.

While the main memory 1706, non-volatile memory 1710, and storage medium 1726 are shown to be a single medium, the terms “storage medium” and “machine-readable medium” should be taken to include a single medium or multiple media that stores one or more sets of instructions 1726. The terms “storage medium” and “machine-readable medium” should also be taken to include any medium that is capable of storing, encoding, or carrying a set of instructions for execution by the processing system 1700.

In general, the routines executed to implement the embodiments of the present disclosure may be implemented as part of an operating system or a specific application, component, program, object, module, or sequence of instructions (collectively referred to as “computer programs”). The computer programs typically comprise one or more instructions (e.g., instructions 1704, 1708, 1728) set at various times in various memories and storage devices in a computing device. When read and executed by the processor 1702, the instructions cause the processing system 1700 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 1710, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 1712 enables the processing system 1700 to mediate data in a network 1714 with an entity that is external to the processing system 1700 through any communication protocol supported by the processing system 1700 and the external entity. The network adapter 1712 can include a network adaptor card, a wireless network interface card, a switch, a protocol converter, a gateway, a bridge, a hub, a receiver, a repeater, or a transceiver that includes an integrated circuit (e.g., enabling communication over Bluetooth or Wi-Fi).

### REMARKS

The foregoing description of various embodiments of the technology has been provided for the purposes of illustration and description. It is not intended to be exhaustive or to limit the claimed subject matter to the precise forms disclosed.

Many modifications and variation will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. A method implemented by an application running on a decentralized computing system, the method comprising:
receiving, by the application from a first client, first input indicative of a request to certify audio data that includes respiratory sounds of an individual;
certifying, by the application, the audio data so as to authorize the application to share analysis of the audio data with entities having an interest in health of the individual;
receiving, by the application from a second client, second input indicative of a request from an entity to access the analysis of the audio data; and
executing, by the application, a smart contract that grants access to the entity to the analysis of the audio data responsive to a determination that the entity satisfies a term contained in the smart contract.

2. The method of claim 1, wherein said certifying comprises:
obtaining, by the application, information regarding a medical device responsible for generating the audio data;
examining, by the application, the information to ensure that the medical device satisfies a criterion; and
producing, by the application, the analysis of the audio file responsive to a determination that the medical device satisfies the criterion.

3. The method of claim 1, wherein said certifying comprises:
examining, by the application, the audio data to ensure that the audio data meets a criterion; and
producing, by the application, the analysis of the audio file responsive to a determination that the audio file satisfies the criterion.

4. The method of claim 3, wherein the predetermined criterion is a minimum duration or a minimum number of breathing cycles.

5. The method of claim 1, wherein the first client resides on a medical device that is responsible for generating the audio data.

6. A method comprising:
generating, by an application running on a decentralized computing system, an interface that is accessible on a first computing device;
receiving, by the application, input indicative of a request to certify an audio file uploaded through the interface on the first computing device, wherein the audio file includes respiratory sounds of an individual recorded by an electronic stethoscope system;
examining, by the application, the audio file to identify a breathing abnormality that is discoverable through analysis of the respiratory sounds; and
forwarding, by the application, information regarding the breathing abnormality to a client executing on a second computing device associated with an entity.

7. The method of claim 6, wherein the entity is a manufacturer of the electronic stethoscope system.

8. The method of claim 6, wherein the entity is a medical facility that is responsible for treating the individual.

9. The method of claim 6, wherein the entity is an insurer involved in paying for treatment of the individual.

10. The method of claim 6, further comprising:
receiving, by the application from the client, second input indicative of a request from the entity to access the information; and
executing, by the application, a smart contract that authorizes access by the entity responsive to a determination that the entity satisfies a term contained in the smart contract;
wherein said forwarding is performed responsive to said executing.

11. A method comprising:
obtaining, by a application, data that is generated by a medical device as part of a diagnostic session involving an individual;
performing, by the application, analysis of the data to derive a diagnostically relevant insight into health of the individual;
establishing, by the application, that an entity is authorized to access the diagnostically relevant insight; and
providing, by the application, the diagnostically relevant insight to the entity.

12. The method of claim 11, wherein said establishing comprises:
executing a smart contract that authorizes access by the entity responsive to a determination that the entity satisfies a term contained in the smart contract.

13. The method of claim 11, wherein said establishing comprises:
determining that the diagnostically relevant insight is to be shared with the entity based on information related to the individual, the diagnostic session, or the medical device, and
confirming that a digital certificate establishing authenticity of the entity is valid.

14. The method of claim 11, further comprising:
recording, by the application, evidence of the diagnostically relevant insight being derived as a transaction on a digital ledger maintained by a decentralized computing system on which the application runs.

15. The method of claim 11, further comprising:
receiving, by the application, input indicative of a request from the entity to access the diagnostically relevant insight;
wherein said establishing is performed responsive to said receiving.

16. The method of claim 15, further comprising:
recording, by the application, evidence of the diagnostically relevant insight being provided as a transaction on a digital ledger maintained by a decentralized computing system on which the application runs.

17. The method of claim 11, wherein the medical device is an electronic stethoscope system configured to transmit the data to the application over the course of the diagnostic session.

18. A method comprising:
obtaining information regarding a medical device;
completing a first validation procedure by executing a first smart contract that validates the medical device responsive to a determination that, based on analysis of the information, the medical device satisfies a first term contained in the first smart contract;
obtaining data generated by the medical device over the course of a diagnostic session involving a patient; and
completing a second validation procedure by executing a second smart contract that validates the medical device responsive to a determination that, based on analysis of the data, the medical device satisfies a second term contained in the second smart contract.

19. The method of claim 18, wherein the information includes an identifier, a dynamic range, a frequency response, an amplification amplitude, a firmware version, or an application version of the medical device.

20. The method of claim 18, further comprising:
provisioning, responsive to completing the first validation procedure, the medical device with a first digital certificate; and
provisioning, responsive to completing the second validation procedure, the medical device with a second digital certificate.
